(19) United States
(12) Patent Application Publication
(10) Pub. No.: US 2024/0228453 A1
(43) Pub. Date: Jul. 11, 2024

(54) CATALYST, APPLICATION THEREOF, AND METHOD FOR PREPARING 2,5-FURANEDICARBOXYLIC ACID BY CATALYZING 5-HYDROMETHYLFURFURAL IN BASE-FREE CONDITION

(71) Applicant: Xiamen University, Xiamen (CN)

(72) Inventors: Xing TANG, Xiamen (CN); Weizhen XIE, Xiamen (CN); Lu LIN, Xiamen (CN); Xianhai ZENG, Xiamen (CN); Yong SUN, Xiamen (CN); Zheng LI, Xiamen (CN); Shuliang YANG, Xiamen (CN)

(21) Appl. No.: 18/544,186

(22) Filed: Dec. 18, 2023

(30) Foreign Application Priority Data
Jan. 6, 2023 (CN) 202310017713.8

(51) Int. Cl. C07D 307/68; B01J 6/00; B01J 23/42; B01J 23/44; B01J 23/46; B01J 27/25; B01J 37/00; B01J 37/04; B01J 37/18

(52) U.S. Cl. CPC C07D 307/68; B01J 6/001; B01J 23/42; B01J 23/44; B01J 23/462; B01J 27/25; B01J 37/0036; B01J 37/04; B01J 37/18

(57) ABSTRACT

A catalyst, an application, and a method for preparing 2,5-furanedicarboxylic acid by catalyzing 5-hydroxymethylfurfural in a base-free condition, which include a catalyst having the formula A/MnₐB_{b}Oₓ-yVC, wherein A is Pt, Ru, Pd, or Au, B is Co, Ce, Cu, or Ni, a mole ratio of a and b is 1.5-14, and y=0.0-0.4.

### RELATED APPLICATIONS

This application claims priority to Chinese patent application 202310017713.8, filed on Jan. 6, 2023. Chinese patent application 202310017713.8 is incorporated herein by reference.

### FIELD OF THE DISCLOSURE

The present disclosure relates to a metal catalyst supported by an Mn-based bimetallic oxide enriched with oxygen vacancies, an application thereof, and a method for preparing 2,5-furanedicarboxylic acid by catalyzing 5-hydroxymethylfurfural in a base-free condition.

### BACKGROUND OF THE DISCLOSURE

Renewable and unique biomass-derived platform chemicals are updated into fuels and valuable chemicals. Not only is dependence on non-renewable depleted petroleum resources reduced, but an interesting path is also found for new materials. In recent years, 2,5-furanedicarboxylic acid (FDCA), which can be fabricated from cellulose via selective oxidation of a biomass-derived platform chemical 5-hydroxymethylfurfural (HMF), has received widespread attention. FDCA-based polyester (PEF) exhibits better barrier properties (02, CO₂) and tensile modulus than terephthalic acid-based polyester (PET). Therefore, the FDCA can be used as a green substitute for terephthalic acid (TPA).

In metal-based catalytic systems, such as using noble-metal catalysts (e.g., Au, Pt, Pd, and Ru) and non-noble-metal catalysts (e.g., Mn, Co, and Ce), the FDCA with high yields (95-99%) can be achieved from the oxidation of HMF (Z. Zhang, K. Deng, ACS Catalog. 2015, 5, 6529-6544; H. Liu, X. Cao, T. Wang, J. Wei, X. Tang, X. Zeng, Y. Sun, T. Lei, S. Liu, L. Lin, J. Ind. Eng. Chem. 2019, 77, 209-214; H. Liu, W. Li, M. Zuo, X. Tang, X. Zeng, Y. Sun, T. Lei, H. Fang, T. Li, L. Lin, Ind. Eng. Chem. Res. 2020, 59, 4895-4904). However, a long reaction time (9-14 hours) and a very low substrate concentration (0.5-2.1 wt %) are required for a majority of Pt-based catalysts to ensure a high FDCA yield (>95%) under a mild and base-free reaction condition (ChemCatChem 2015, 7, 2853-2863; Green Chem., 2016, 18, 1597-1604; Green Process. Synth. 5 (2016) 353-364; Applied Catalysis A, General 555 (2018) 98-107; Applied Catalysis A: General 526 (2016)1-8), which obviously cannot meet needs of industrial production of the FDCA. To be specific, larger reactors are needed if the substrate concentration is low in these cases, which results in an increase of equipment investment and an energy-intensive separation and purification of products. In addition, most catalytic systems require pure oxygen as an oxidant. The employment of ubiquitous air as an oxygen source is more desirable from the perspective of economy. In this regard, an establishment of a catalytic system for a preparation of the FDCA from the HMF at high concentrations is highly desirable for a large-scale production of the FDCA.

However, special chemical properties of the HMF and the FDCA, such as poor stability of the HMF and low solubility of the FDCA in most solvents, results in a challenging task for the preparation of the FDCA from a concentrated HMF solution (Green Chem. 19 (2017) 996-1004.). In this context, Hensen and Nakajima first found that HMF acetal derivative (PD-HMF), derived from a reaction of the HMF with 1,3-propanediol, showed an excellent chemical stability as compared to the HMF, which brought about a desirable FDCA yield of 94% from concentrated PD-HMF (10 wt %) in the presence of Na₂CO₃ in water (140° C., 15 hours, 0.5 MPa O₂) (Angew. Chem. Int. Ed. 57 (2018) 8235-8239.). In contrast, the FDCA yield of only 28% was achieved from the HMF (10 wt %) at the identical reaction conditions, which is largely attributed to the instability of HMF as compared to its acetal derivative (Angew. Chem. Int. Ed. 57 (2018) 8235-8239.).

A reaction solvent system significantly affects the reaction pathway and efficiency of HMF conversion, especially in the case of high substrate concentration (Chem. Sus. Chem. 9 (2016) 133-155.). However, few studies focus on the solvent effect of an HMF oxidation process. Many works have proved that the HMF is more stable in an organic solvent or organic solvent/H₂O mixtures than in pure water (Green Chem. 16 (2014) 2015-2026.). For instance, Won et al., found that an introduction of a mixed solvent of γ-valerolactone (GVL) and water could greatly improve the solubility of the FDCA when comparing with that in pure water or GVL, which enables catalytic conversion of the HMF to the FDCA at relatively high concentration (7.5 wt % HMF) without base additive (Sci. Adv. 4 (2018) p9722.). However, the oxidation of the HMF at a concentration greater than 7.5 wt % did not perform in the mixed solution of the GVL and the H₂O, and a long reaction time (20 hours) was required to ensure a high FDCA yield. Interestingly, a DMSO-H₂O solvent system allowed the production of the FDCA from 10 wt % HMF over Ru/C, and a desirable FDCA yield was up to 93% in a base environment (J Ind Eng Chem. 77 (2019) 209-214.). Nevertheless, a reaction time of 12 hours was still needed for the catalytic conversion of the concentrated HMF (10 wt %) in the DMSO-H₂O solvent system. However, the utilisation of organic solvents makes the reaction system environmentally unfriendly as well as brings difficulties in subsequent separation and purification of the products. Therefore, there is an urgent need to develop and design a catalyst that can efficiently catalyze the concentrated HMF to produce the FDCA in an aqueous system. It is well known that the dehydrogenation of the HMF, as well as its semi-acetal intermediates, requires highly active lattice oxygen sites. The formation of the semi-acetal intermediates could be greatly facilitated under a base condition. A challenge for the base-free oxidation that needs to be solved is that the dehydrogenation of the HMF as well as its semi-acetal intermediates need to be efficiently catalyzed without promoting the formation of the semi-acetal intermediates. Therefore, it is urgent to develop oxidation catalysts with higher active lattice oxygen sites.

### BRIEF SUMMARY OF THE DISCLOSURE

An objective of the present disclosure is to provide a catalyst, an application thereof, and a method for preparing 2,5-furanedicarboxylic acid by catalyzing 5-hydroxymethylfurfural in a base-free condition to solve the deficiencies of the existing techniques, and the aforementioned problems in the background are therefore resolved.

A first technical solution of the present disclosure to solve the technical problem is as follows:

A method for preparing 2,5-furanedicarboxylic acid by catalyzing 5-hydroxymethylfurfural in a base-free condition comprises the following steps:

Synthesizing the 2,5-furanedicarboxylic acid by catalyzing the 5-hydroxymethylfurfural using a catalyst in the base-free condition with a solvent of water using air or oxygen as an oxygen source, and a reaction time is 0.5-21 hours; and the catalyst is a metal catalyst supported by an Mn-based bimetallic oxide enriched with oxygen vacancies, a formula of the catalyst is A/MnₐB_{b}Oₓ-yVC, wherein A is at least one of Pt, Ru, Pd, or Au, B is at least one of Co, Ce, Cu, or Ni, a mole ratio of a and b is 1.5-14, and y=0.0-0.4.

In a preferred embodiment, a reaction pressure is 0.2-4.0 MPa, a temperature of a reaction autoclave for the method is 80-130° C., and the reaction time is 1-21 hours.

In a preferred embodiment, preparing the catalyst comprises the following steps:
1) mixing and grinding a precursor manganese nitrate, metal nitrate, and ascorbic acid, then calcining at 200-500° C. for 2 hours to obtain a carrier with the Mn-based bimetallic oxide enriched with oxygen vacancies, wherein the metal nitrate is at least one of cobalt nitrate, cerium nitrate, copper nitrate, or nickel nitrate, a molar ratio of the precursor manganese nitrate and the metal nitrate is 1.5-14:1, and a molar ratio of the ascorbic acid and a sum of the precursor manganese nitrate and the metal nitrate is 0-0.4:1; and
2) adding at least one of hexahydrate chloroplatinic acid, trihydrate ruthenium chloride, palladium chloride, trihydrate chloroauric acid and the Mn-based bimetallic oxide carrier to deionized water, stirring to be dispersed to even, and reducing to obtain the catalyst.

In a preferred embodiment, in the step 2), the reducing to obtain the catalyst comprises adding a sodium borohydride solution (e.g., a newly prepared sodium borohydride solution), continually stirring for 2 hours, then filtering, and drying the catalyst.

In a preferred embodiment, the reducing to obtain the catalyst in the step 2) comprises drying by evaporating water, then calcinating at 500° C. for 4 hours, and then reducing at 500° C. in a hydrogen atmosphere for 1 hour.

A second technical solution of the present disclosure to solve the technical problem is as follows:

A metal catalyst supported by an Mn-based bimetallic oxide enriched with oxygen vacancies, wherein a metal of the metal catalyst is A, and a formula of the metal catalyst is A/MnₐB_{b}Oₓ-yVC, wherein A is at least one of Pt, Ru, Pd, or Au, B is at least one of Co, Ce, Cu, or Ni, a mole ratio of a and b is 1.5-14, and y=0.0-0.4.

A third technical solution of the present disclosure to solve the technical problem is as follows:

An application of the metal catalyst supported by the Mn-based bimetallic oxide enriched with the oxygen vacancies comprises preparing 2,5-furanedicarboxylic acid by catalyzing 5-hydroxymethylfurfural using the metal catalyst supported by the Mn-based bimetallic oxide enriched with the oxygen vacancies.

In a preferred embodiment, the preparing 2,5-furanedicarboxylic acid by catalyzing 5-hydroxymethylfurfural using the metal catalyst supported by the Mn-based bimetallic oxide enriched with the oxygen vacancies comprises: mixing the 5-hydroxymethylfurfural and water solvent and placing in a reaction kettle; adding the metal catalyst under a base-free condition; and sealing the reaction kettle, and filling with air or oxygen, wherein a pressure is 0.2-4.0 MPa, a temperature of the reaction kettle is 80-130° C., and a reaction time is 0.5-21 hours.

In a preferred embodiment, the pressure is 0.5-2.5 MPa, and the reaction time 0.5-2 hours.

In a preferred embodiment, the reaction time is 0.5-2 hours.

In a preferred embodiment, y is 0.1-0.4.

In a preferred embodiment, a loading amount of A is 1-5 wt %.

Compared with the background, the technical solution has the following advantages:
(1) In the present disclosure, a metal catalyst supported by the Mn-based bimetallic oxide enriched with oxygen vacancies is designed and synthesized. The problem of the existing HMF oxidation for conducting at very low substrate concentration (0.5-2.1 wt %) and requiring a large amount of base additives can be overcame;
(2) The present disclosure firstly prepares the Mn-based bimetallic oxide enriched with oxygen vacancies through a simple solid-phase grinding calcination process assisted by ascorbic acid (VC) and then increases a concentration of the oxygen vacancies of the surface of the catalyst by loading noble metals to obtain a catalyst with efficient catalytic activity for the oxidation of the 5-hydroxymethylfurfural.
(3) The metal catalyst supported by the Mn-based bimetallic oxide enriched with the oxygen vacancies prepared by the present disclosure has the highest catalytic activity for preparing 2,5-furanedicarboxylic acid by the oxidation of the 5-hydroxymethylfurfural under a mild and base-free reaction condition in water as a green solvent, and the metal catalyst is also better than other catalysts based on a same metal when other metals of the other catalysts are loaded;
(4) The present disclosure establishes a green and efficient catalytic system for a catalytic preparation of the 2,5-furanedicarboxylic acid under the base-free condition to overcome the problem of the existing HMF oxidation for conducting at the very low substrate concentration (0.5-2.1 wt %) and requiring the large amount of base additives. Air can be directly used as oxygen source, oxidation costs are effectively reduced, a reaction time is greatly shortened, and an excellent conversion rate is achieved;
(5) When the oxidation of the 5-hydroxymethylfurfural is performed under a base-free catalytic condition at 80-130° C. and 0.2-4.0 MPa of air or pure oxygen for 1-2 hours, a yield of 2,5-furandicarboxylic acid can reach 95%. Therefore, the present disclosure effectively solves the problem of other currently reported Pt-based catalysts in which a reaction time of 9-14 hours is usually required to achieve similar catalytic effects under the base-free condition. Moreover, FDCA yield of 83%-93% is achieved from oxidation of concentrated pure HMF (5-40 wt %) in pure H₂O under base-free conditions. Unexpectedly, FDCA yield above 90% is obtained from the oxidation of the concentrated crude HMF (1-10 wt %) in the pure H₂O under the base-free conditions. As far as we know, there is no previous report on an efficient preparation of the FDCA from such high HMF concentration, especially in the oxidation of the concentrated crude HMF.

### BRIEF DESCRIPTION OF THE DRAWING

The present disclosure will be further described in combination with the accompanying drawings and embodiments.

FIGS. 1A and 1B show an electron paramagnetic resonance (EPR) spectrum of oxygen vacancy (Oᵥ) of a catalyst.

FIG. 2 shows O1s in an X-ray photoelectron high-resolution spectroscopy (XPS) spectrum of the catalyst.

FIGS. 3A and 3B show a H₂ temperature-programmed reduction (H₂-TPR) spectrum of the catalyst.

### DETAILED DESCRIPTION OF THE EMBODIMENTS

In the following embodiments or comparison embodiments 1-48, a catalyst is described as Pt (z wt %)/MnₐCo_{b}Oₓ-yVC-w ° C., where z represents a loading amount of Pt (unless otherwise defined, a default value is 2.2 wt %); in which a and b represents a molar ratio of Mn and Co in the catalyst, y represents a molar ratio of the VC and a total metal content (i.e., a molar ratio of the VC and (Mn+Co)), and w represents a calcination temperature of Mn-based bimetallic oxide, which is 200° C.-500° C. (unless otherwise defined, a default value is 200° C.).

The catalyst is used to prepare 2,5-furanedicarboxylic acid from the oxidation of 5-hydroxymethylfurfural under base-free conditions. A reaction path is as follows:

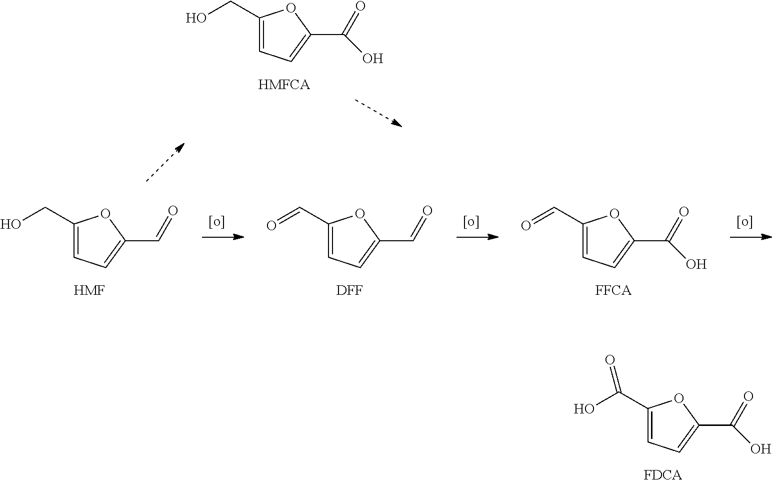

Note: 2,5-Diformylfuran (DFF); 5-hydroxymethyl-2-furancarboxylic acid (HMFCA); 5-formyl-2-furancarboxylic acid (FFCA).

### Embodiment 1

A preparation of a catalyst with platinum nanoparticles supported by manganese oxide: first, 10.0116 g of manganese nitrate tetrahydrate (Mn(NO₃)₂·4H₂O) is manually ground for 5 minutes, then calcined at 200° C. for 2 hours under air atmosphere (e.g., 1 atm) to obtain an oxide MnOₓ. Second, 1.0019 g of the MnOₓ, 0.0884 g of hexahydrate chloroplatinic acid (H₂PtCl₆·6H₂O), and 20.1762 g of deionized water are added to a beaker (100 mL) to obtain a first mixture, and the first mixture is stirred for 0.5 hours. 0.0645 g of sodium borohydride is dissolved in 15.0292 g of the deionized water to prepare a sodium borohydride solution (a molar ratio of NaBH₄ and H₂PtCl₆·6H₂O is 10). The sodium borohydride solution is then added in drops under magnetic stirring (500 revolutions per minute (rpm)) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese oxide (called Pt/MnOₓ).

Oxidation reaction of 5-hydroxymethyl furfural is as follows: 0.0309 g of the 5-hydroxymethyl furfural, 0.0403 g of the Pt/MnOₓ, and 3.0245 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 1.

### Embodiment 2

A preparation of a catalyst with platinum nanoparticles supported by cobalt oxide: first, 10.0052 g of cobalt nitrate hexahydrate (Co(NO₃)₂·6H₂O) is manually ground for 5 minutes, then calcined at 200° C. for 2 hours under air atmosphere (e.g., 1 atm) to obtain an oxide CoOₓ. Second, 1.0036 g of the CoOₓ, 0.0889 g of hexahydrate chloroplatinic acid (H₂PtCl₆·6H₂O), and 20.0758 g of deionized water are added to a beaker (100 mL) to obtain a first mixture, and the first mixture is stirred for 0.5 hours. 0.0645 g of sodium borohydride is dissolved in 15.0588 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by cobalt oxide (called Pt/CoOₓ).

Oxidation reaction of 5-hydroxymethyl furfural is as follows: 0.0323 g of the 5-hydroxymethyl furfural, 0.0408 g of the Pt/CoOₓ, and 3.0020 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 2.

### Embodiment 3

A preparation of a catalyst with platinum nanoparticles supported by manganese oxide: first, 10.0273 g of manganese nitrate tetrahydrate (Mn(NO₃)₂·4H₂O) and 1.4042 g of ascorbic acid (VC) are manually ground for 5 minutes, then calcined at 200° C. for 2 hours under air atmosphere (e.g., 1 atm) to obtain an oxide MnOₓ-0.2VC. Second, 1.0032 g of the MnOₓ-0.2VC, 0.0885 g of hexahydrate chloroplatinic acid (H₂PtCl₆·6H₂O), and 20.0354 g of deionized water are added to a beaker (100 mL) to obtain a first mixture, and the first mixture is stirred for 0.5 hours. 0.0643 g of sodium borohydride is dissolved in 15.0300 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese oxide (called Pt/MnOₓ-0.2VC).

Oxidation reaction of 5-hydroxymethyl furfural is as follows: 0.0322 g of the 5-hydroxymethyl furfural, 0.0400 g of the Pt/MnOₓ-0.2VC, and 3.0029 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 3.

### Embodiment 4

A preparation of a catalyst with platinum nanoparticles supported by cobalt oxide: first, 10.0090 g of cobalt nitrate hexahydrate (Co(NO₃)₂·6H₂O) and 1.2117 g of ascorbic acid (VC) are manually ground for 5 minutes, then calcined at 200° C. for 2 hours under air atmosphere (e.g., 1 atm) to obtain an oxide CoOₓ-0.2VC. Second, 1.0013 g of the CoOₓ-0.2VC, 0.0887 g of hexahydrate chloroplatinic acid (H₂PtCl₆·6H₂O), and 20.0795 g of deionized water are added to a beaker (100 mL) to obtain a first mixture, and the first mixture is stirred for 0.5 hours. 0.0648 g of sodium borohydride is dissolved in 15.1019 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by cobalt oxide (called Pt/CoOₓ-0.2VC).

Oxidation reaction of 5-hydroxymethyl furfural is as follows: 0.0302 g of the 5-hydroxymethyl furfural, 0.0402 g of the Pt/CoOₓ-0.2VC, and 3.0289 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 4.

### Embodiment 5

A preparation of a catalyst with platinum nanoparticles supported by manganese-cobalt oxide: first, 9.7416 g of manganese nitrate tetrahydrate (Mn(NO₃)₂·4H₂O), 0.8061 g of cobalt nitrate hexahydrate (Co(NO₃)₂·6H₂O), and 1.4629 g of ascorbic acid (VC) are manually ground for 5 minutes, then calcined at 200° C. for 2 hours under air atmosphere (e.g., 1 atm) to obtain an oxide Mn₁₄Co₁Oₓ-0.2VC. Second, 1.0027 g of the Mn₁₄Co₁Oₓ-0.2VC, 0.0889 g of hexahydrate chloroplatinic acid (H₂PtCl₆·6H₂O), and 20.2250 g of deionized water are added to a beaker (100 mL) to obtain a first mixture, and the first mixture is stirred for 0.5 hours. 0.0640 g of sodium borohydride is dissolved in 15.3397 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₁₄Co₁Oₓ-0.2VC).

Oxidation reaction of 5-hydroxymethyl furfural is as follows: 0.0300 g of the 5-hydroxymethyl furfural, 0.0406 g of the Pt/Mn₁₄Co₁Oₓ-0.2VC, and 3.0221 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 5.

### Embodiment 6

A preparation of a catalyst with platinum nanoparticles supported by manganese-cobalt oxide: first, 9.6077 g of manganese nitrate tetrahydrate (Mn(NO₃)₂·4H₂O), 0.9281 g of cobalt nitrate hexahydrate (Co(NO₃)₂·6H₂O), and 1.4629 g of ascorbic acid (VC) are manually ground for 5 minutes, then calcined at 200° C. for 2 hours under air atmosphere (e.g., 1 atm) to obtain an oxide Mn₁₂Co₁Oₓ-0.2VC. Second, 1.0005 g of the Mn₁₂Co₁Oₓ-0.2VC, 0.0887 g of hexahydrate chloroplatinic acid (H₂PtCl₆·6H₂O), and 20.2085 g of deionized water are added to a beaker (100 mL) to obtain a first mixture, and the first mixture is stirred for 0.5 hours. 0.0645 g of sodium borohydride is dissolved in 15.1055 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₁₂Co₁Oₓ-0.2VC).

Oxidation reaction of 5-hydroxymethyl furfural is as follows: 0.0300 g of the 5-hydroxymethyl furfural, 0.0405 g of the Pt/Mn₁₂Co₁Oₓ-0.2VC, and 3.0472 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 6.

### Embodiment 7

A preparation of a catalyst with platinum nanoparticles supported by manganese-cobalt oxide: first, 9.4762 g of manganese nitrate tetrahydrate (Mn(NO₃)₂·4H₂O), 1.0977 g of cobalt nitrate hexahydrate (Co(NO₃)₂·6H₂O), and 1.4620 g of ascorbic acid (VC) are manually ground for 5 minutes, then calcined at 200° C. for 2 hours under air atmosphere (e.g., 1 atm) to obtain an oxide Mn₁₀Co₁Oₓ-0.2VC. Second, 1.0015 g of the Mn₁₀Co₁Oₓ-0.2VC, 0.0882 g of hexahydrate chloroplatinic acid (H₂PtCl₆·6H₂O), and 20.2923 g of deionized water are added to a beaker (100 mL) to obtain a first mixture, and the first mixture is stirred for 0.5 hours. 0.0640 g of sodium borohydride is dissolved in 15.2198 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₁₀Co₁Oₓ-0.2VC).

Oxidation reaction of 5-hydroxymethyl furfural is as follows: 0.0309 g of the 5-hydroxymethyl furfural, 0.0410 g of the Pt/Mn₁₀Co₁Oₓ-0.2VC, and 3.0559 g of the deionized water are added into a 25 mL autoclave, and 1 MPa air is charged into the reactor. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 7.

### Embodiment 8

A preparation of a catalyst with platinum nanoparticles supported by manganese-cobalt oxide: first, 9.2440 g of manganese nitrate tetrahydrate (Mn(NO₃)₂·4H₂O), 1.3435 g of cobalt nitrate hexahydrate (Co(NO₃)₂·6H₂O), and 1.4624 g of ascorbic acid (VC) are manually ground for 5 minutes, then calcined at 200° C. for 2 hours under air atmosphere (e.g., 1 atm) to obtain an oxide Mn₈Co₁Oₓ-0.2VC. Second, 1.0008 g of the Mn₈Co₁Oₓ-0.2VC, 0.0884 g of hexahydrate chloroplatinic acid (H₂PtCl₆·6H₂O), and 20.3245 g of deionized water are added to a beaker (100 mL) to obtain a first mixture, and the first mixture is stirred for 0.5 hours. 0.0647 g of sodium borohydride is dissolved in 15.2656 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₈Co₁Oₓ-0.2VC).

Oxidation reaction of 5-hydroxymethyl furfural is as follows: 0.0313 g of the 5-hydroxymethyl furfural, 0.0408 g of the Pt/Mn₈Co₁Oₓ-0.2VC, and 3.0020 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 8.

### Embodiment 9

A preparation of a catalyst with platinum nanoparticles supported by manganese-cobalt oxide: first, 8.9339 g of manganese nitrate tetrahydrate (Mn(NO₃)₂·4H₂O), 1.7246 g of cobalt nitrate hexahydrate (Co(NO₃)₂·6H₂O), and 1.4626 g of ascorbic acid (VC) are manually ground for 5 minutes, then calcined at 200° C. for 2 hours under air atmosphere (e.g., 1 atm) to obtain an oxide Mn₆Co₁Oₓ-0.2VC. Second, 1.0021 g of the Mn₆Co₁Oₓ-0.2VC, 0.0885 g of hexahydrate chloroplatinic acid (H₂PtCl₆·6H₂O), and 20.2499 g of deionized water are added to a beaker (100 mL) to obtain a first mixture, and the first mixture is stirred for 0.5 hours. 0.0644 g of sodium borohydride is dissolved in 15.2877 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₆Co₁Oₓ-0.2VC).

Oxidation reaction of 5-hydroxymethyl furfural is as follows: 0.0298 g of the 5-hydroxymethyl furfural, 0.0405 g of the Pt/Mn₆Co₁Oₓ-0.2VC, and 3.0302 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 9.

### Embodiment 10

A preparation of a catalyst with platinum nanoparticles supported by manganese-cobalt oxide: first, 8.3404 g of manganese nitrate tetrahydrate (Mn(NO₃)₂·4H₂O), 2.4165 g of cobalt nitrate hexahydrate (Co(NO₃)₂·6H₂O), and 1.4631 g of ascorbic acid (VC) are manually ground for 5 minutes, then calcined at 200° C. for 2 hours under air atmosphere (e.g., 1 atm) to obtain an oxide Mn₄Co₁Oₓ-0.2VC. Second, 1.0018 g of the Mn₄Co₁Oₓ-0.2VC, 0.0887 g of hexahydrate chloroplatinic acid (H₂PtCl₆·6H₂O), and 20.1669 g of deionized water are added to a beaker (100 mL) to obtain a first mixture, and the first mixture is stirred for 0.5 hours. 0.0643 g of sodium borohydride is dissolved in 15.1830 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₄Co₁Oₓ-0.2VC).

Oxidation reaction of 5-hydroxymethyl furfural is as follows: 0.0302 g of the 5-hydroxymethyl furfural, 0.0398 g of the Pt/Mn₄Co₁Oₓ-0.2VC, and 3.0141 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 10.

### Embodiment 11

A preparation of a catalyst with platinum nanoparticles supported by manganese-cobalt oxide: first, 7.8125 g of manganese nitrate tetrahydrate (Mn(NO₃)₂·4H₂O), 3.0195 g of cobalt nitrate hexahydrate (Co(NO₃)₂·6H₂O), and 1.4621 g of ascorbic acid (VC) are manually ground for 5 minutes, then calcined at 200° C. for 2 hours under air atmosphere (e.g., 1 atm) to obtain an oxide Mn₃Co₁Oₓ-0.2VC. Second, 1.0020 g of the Mn₃Co₁Oₓ-0.2VC, 0.0892 g of hexahydrate chloroplatinic acid (H₂PtCl₆·6H₂O), and 20.0968 g of deionized water are added to a beaker (100 mL) to obtain a first mixture, and the first mixture is stirred for 0.5 hours. 0.0649 g of sodium borohydride is dissolved in 15.1293 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₃Co₁Oₓ-0.2VC).

Oxidation reaction of 5-hydroxymethyl furfural is as follows: 0.0329 g of the 5-hydroxymethyl furfural, 0.0406 g of the Pt/Mn₃Co₁Oₓ-0.2VC, and 3.0138 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 11.

### Embodiment 12

A preparation method of the Pt/Mn₁₀Co₁Oₓ-0.2VC is the same as Embodiment 7.

Oxidation reaction of 5-hydroxymethyl furfural is as follows: 0.0302 g of the 5-hydroxymethyl furfural, 0.0602 g of the Pt/Mn₁₀Co₁Oₓ-0.2VC, and 3.0353 g of the deionized water are added into a 25 mL autoclave, and 1.5 MPa air is charged into the reactor. Afterward, the reaction mixture is stirred at 120° C. for 1.5 hours. Then the 25 mL autoclave is cooled to room temperature (e.g., 20-25° C.), and the reaction solution is analyzed quantitatively. The test results are listed in Table 1 as No. 12.

### Embodiment 13

A preparation method of the Pt/Mn₁₀Co₁Oₓ-0.2VC is the same as Embodiment 7.

Oxidation reaction of 5-hydroxymethyl furfural is as follows: 0.0302 g of the 5-hydroxymethyl furfural, 0.0602 g of the Pt/Mn₁₀Co₁Oₓ-0.2VC, and 3.0353 g of the deionized water are added into a 25 mL autoclave, and 0.2 MPa oxygen is charged into the 25 mL autoclave. Afterward, the reaction mixture is stirred at 120° C. for 1 hour. Then the 25 mL autoclave is cooled to room temperature (e.g., 20-25° C.), and the reaction solution is analyzed quantitatively. The test results are listed in Table 1 as No. 13.

### Embodiment 14

A preparation of a catalyst with platinum nanoparticles supported by manganese-cobalt oxide: first, 270.08 g of an aqueous solution of manganese nitrate (Mn(NO₃)₂, 50 wt %), 21.97 g of cobalt nitrate hexahydrate (Co(NO₃)₂·6H₂O), and 29.26 g of ascorbic acid (VC) are manually ground for 5 minutes, then calcined at 200° C. for 2 hours under air atmosphere (e.g., 1 atm) to obtain an oxide Mn₁₀Co₁Oₓ-0.2VC-LS. Second, 21.0034 g of the Mn₁₀Co₁Oₓ-0.2VC-LS, 1.8590 g of hexahydrate chloroplatinic acid (H₂PtCl₆·6H₂O), and 420.64 g of deionized water are added to a beaker (2000 mL) to obtain a first mixture, and the first mixture is stirred for 2 hours. 1.3531 g of sodium borohydride is dissolved in 315.0457 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 12 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₁₀Co₁Oₓ-0.2VC-LS).

Oxidation reaction of 5-hydroxymethyl furfural is as follows: 0.0305 g of the 5-hydroxymethyl furfural, 0.0611 g of the Pt/Mn₁₀Co₁Oₓ-0.2VC-LS, and 3.0432 g of the deionized water are added into a 25 mL autoclave, and 1.5 MPa air is charged into the 25 mL autoclave. Afterward, the reaction mixture is stirred at 120° C. for 1.5 hours. Then the 25 mL autoclave is cooled to room temperature (e.g., 20-25° C.), and the reaction solution is analyzed quantitatively. The test results are listed in Table 1 as No. 14.

### Embodiment 15

A preparation method of the Pt/Mn₁₀Co₁Oₓ-0.2VC is the same as Embodiment 7.

Oxidation reaction of 5-hydroxymethyl furfural is as follows: 0.0306 g of the 5-hydroxymethyl furfural, 0.0402 g of the Pt/Mn₁₀Co₁Oₓ-0.2VC, and 3.0275 g of the deionized water are added into a 25 mL autoclave, and 1 MPa oxygen is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 15.

### Embodiments 16-19

A preparation of a catalyst with platinum nanoparticles supported by manganese-cobalt oxide: first, 9.4762 g of manganese nitrate tetrahydrate (Mn(NO₃)₂·4H₂O), 1.0977 g of cobalt nitrate hexahydrate (Co(NO₃)₂·6H₂O), and 0 g, 0.7322 g, 2.1924 g, or 2.9237 g of ascorbic acid (VC) are manually ground for 5 minutes, then calcined at 200° C. for 2 hours under air atmosphere (e.g., 1 atm) to sequentially obtain four oxides, Mn₁₀Co₁Oₓ, Mn₁₀Co₁Oₓ-0.1VC, Mn₁₀Co₁Oₓ-0.3VC, or Mn₁₀Co₁Oₓ-0.4VC. Second, a preparation of the catalyst with the platinum nanoparticles supported by the four oxides is as follows: 1.0000 g of the four oxides, 0.0889 g of hexahydrate chloroplatinic acid (H₂PtCl₆·6H₂O), and 20.0000 g of deionized water are added to a beaker (100 mL) to obtain a first mixture, and the first mixture is stirred for 0.5 hours. 0.0644 g of sodium borohydride is dissolved in 15.0000 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (sequentially called Pt/Mn₁₀Co₁Oₓ, Pt/Mn₁₀Co₁Oₓ-0.1VC, Pt/Mn₁₀Co₁Oₓ-0.3VC, or Pt/Mn₁₀Co₁Oₓ-0.4VC).

Oxidation reaction of 5-hydroxymethyl furfural is as follows: 0.0300 g of the 5-hydroxymethyl furfural, 0.0400 g of the Pt/Mn₁₀Co₁Oₓ-(0-0.4)VC, and 3.0000 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are sequentially listed in Table 1 as Nos. 16-19.

### Embodiments 20-22

A preparation of a catalyst with platinum nanoparticles supported by manganese-cobalt oxide: first, 9.4762 g of manganese nitrate tetrahydrate (Mn(NO₃)₂·4H₂O), 1.0977 g of cobalt nitrate hexahydrate (Co(NO₃)₂·6H₂O), and 1.4620 g of ascorbic acid (VC) are manually ground for 5 minutes, then respectively calcined at 300° C., 400° C., or 500° C. for 2 hours under air atmosphere (e.g., 1 atm) to sequentially obtain three oxides, Mn₁₀Co₁Oₓ-0.2VC-300° C., Mn₁₀Co₁Oₓ-0.2VC-400° C., or Mn₁₀Co₁Oₓ-0.2VC-500° C. Second, a preparation of the catalyst with the platinum nanoparticles supported by the three oxides is as follows: 1.0000 g of the three oxides, 0.0889 g of hexahydrate chloroplatinic acid (H₂PtCl₆·6H₂O), and 20.0000 g of deionized water are added to a beaker (100 mL) to obtain a first mixture, and the first mixture is stirred for 0.5 hours. 0.0644 g of sodium borohydride is dissolved in 15.0000 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (sequentially called Pt/Mn₁₀Co₁Oₓ-0.2VC-300° C., Pt/Mn₁₀Co₁Oₓ-0.2VC-400° C., or Pt/Mn₁₀Co₁Oₓ-0.2VC-500° C.).

Oxidation reaction of 5-hydroxymethyl furfural is as follows: 0.0300 g of the 5-hydroxymethyl furfural, 0.0400 g of the Pt/Mn₁₀Co₁Oₓ-0.2VC-(300-500° C.), and 3.0000 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are sequentially listed in Table 1 as Nos. 20-22.

### Embodiments 23-26

A preparation method of the Mn₁₀Co₁Oₓ-0.2VC is the same as Embodiment 7.

A preparation of the catalyst with the platinum nanoparticles supported by the oxide Mn₁₀Co₁Oₓ-0.2VC is as follows: 1.0000 g of the oxide, 0.0356 g, 0.0634 g, 0.1174 g, or 0.1430 g of hexahydrate chloroplatinic acid (H₂PtCl₆·6H₂O), and 20.0000 g of deionized water are added to a beaker (100 mL) to obtain a first mixture, and the first mixture is stirred for 0.5 hours. 0.0644 g of sodium borohydride is dissolved in 15.0000 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (sequentially called Pt (1.1 wt %)/Mn₁₀Co₁Oₓ-0.2VC, Pt (1.8 wt %)/Mn₁₀Co₁Oₓ-0.2VC, Pt (3.3 wt %)/Mn₁₀Co₁Oₓ-0.2VC, or Pt (4.4 wt %)/Mn₁₀Co₁Oₓ-0.2VC).

Oxidation reaction of 5-hydroxymethyl furfural is as follows: 0.0300 g of the 5-hydroxymethyl furfural, 0.0400 g of the Pt (1.1-4.4 wt %)/Mn₁₀Co₁Oₓ-0.2VC, and 3.0000 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as Nos. 23-26.

### Embodiment 27

A preparation of a catalyst with ruthenium nanoparticles supported by manganese-cobalt oxide: first, 6.2775 g of manganese nitrate tetrahydrate (Mn(NO₃)₂·4H₂O), 4.8020 g of cobalt nitrate hexahydrate (Co(NO₃)₂·6H₂O), and 1.4620 g of ascorbic acid (VC) are manually ground for 5 minutes, then calcined at 200° C. for 2 hours under air atmosphere (e.g., 1 atm) to obtain an oxide Mn₃Co₂Oₓ-0.2VC. Second, 0.5043 g of the Mn₃Co₂Oₓ-0.2VC, 0.0445 g of ruthenium chloride trihydrate (RuCl₃·3H₂O), and 20.1020 g of deionized water are added to a beaker (100 mL) to obtain a first mixture, and the first mixture is stirred for 0.5 hours. 0.0628 g of sodium borohydride is dissolved in 15.1459 g of the deionized water to prepare a sodium borohydride solution (a molar ratio of NaBH₄ and RuCl₃-3H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with ruthenium nanoparticles supported by manganese-cobalt oxide (called Ru/Mn₃Co₂Oₓ-0.2VC).

Oxidation reaction of 5-hydroxymethyl furfural is as follows: 0.0298 g of the 5-hydroxymethyl furfural, 0.0420 g of the Ru/Mn₃Co₂Oₓ-0.2VC, and 3.0268 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 27.

### Embodiments 28

A preparation method of the Mn₃Co₂Oₓ-0.2VC is the same as Embodiment 27.

A preparation of the catalyst with the noble-metal platinum nanoparticles supported by the oxide Mn₃Co₂Oₓ-0.2VC is as follows: 1.0018 g of the oxide Mn₃Co₂Oₓ-0.2VC, 0.0891 g of hexahydrate chloroplatinic acid (H₂PtCl₆·6H₂O), and 20.3243 g of deionized water are added to a beaker (100 mL) to obtain a first mixture, and the first mixture is stirred for 0.5 hours. 0.0656 g of sodium borohydride is dissolved in 15.0667 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt/Mn₃Co₂Oₓ-0.2VC).

Oxidation reaction of 5-hydroxymethyl furfural is as follows: 0.0303 g of the 5-hydroxymethyl furfural, 0.0415 g of the Pt/Mn₃Co₂Oₓ-0.2VC, and 3.0394 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 28.

### Embodiments 29

A preparation method of the Mn₃Co₂Oₓ-0.2VC is the same as Embodiment 27.

A preparation of the catalyst with the noble-metal platinum nanoparticles supported by the oxide Mn₃Co₂Oₓ-0.2VC is as follows: 1.0083 g of the oxide Mn₃Co₂Oₓ-0.2VC, 0.0423 g of trihydrate chloroauric acid (HAuCl₄·3H₂O), and 20.0712 g of deionized water are added to a beaker (100 mL) to obtain a first mixture, and the first mixture is stirred for 0.5 hours. 0.0393 g of sodium borohydride is dissolved in 15.0784 g of the deionized water to prepare a sodium borohydride solution (a molar ratio of NaBH₄ and HAuCl₄·3H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with gold nanoparticles supported by manganese-cobalt oxide (called Au/Mn₃Co₂Oₓ-0.2VC).

Oxidation reaction of 5-hydroxymethyl furfural is as follows: 0.0301 g of the 5-hydroxymethyl furfural, 0.0670 g of the Au/Mn₃Co₂Oₓ-0.2VC, and 3.0559 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 29.

### Embodiments 30

A preparation method of the Mn₃Co₂Oₓ-0.2VC is the same as Embodiment 27.

A preparation of the catalyst with the noble-metal palladium nanoparticles supported by the oxide Mn₃Co₂Oₓ-0.2VC is as follows: 1.0079 g of the oxide Mn₃Co₂Oₓ-0.2VC, 0.0346 g of palladium chloride (PdCl₂), and 20.0254 g of deionized water are added to a beaker (100 mL) to obtain a first mixture, and the first mixture is stirred for 0.5 hours. 0.0718 g of sodium borohydride is dissolved in 15.3153 g of the deionized water to prepare a sodium borohydride solution (a molar ratio of NaBH₄ and PdCl₂ is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with palladium nanoparticles supported by manganese-cobalt oxide (called Pd/Mn₃Co₂Oₓ-0.2VC).

Oxidation reaction of 5-hydroxymethyl furfural is as follows: 0.0300 g of the 5-hydroxymethyl furfural, 0.0374 g of the Pd/Mn₃Co₂Oₓ-0.2VC, and 3.0426 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 30.

### Embodiments 31

A preparation method of the Mn₃Co₂Oₓ-0.2VC is the same as Embodiment 27.

A preparation of the catalyst with the noble-metal ruthenium nanoparticles supported by the oxide Mn₃Co₂Oₓ-0.2VC is as follows: 1.0009 g of the oxide Mn₃Co₂Oₓ-0.2VC, 0.0878 g of ruthenium chloride trihydrate (RuCl₃·3H₂O), and 20.1596 g of deionized water are added to a beaker (100 mL) to obtain a first mixture, and the first mixture is stirred for 0.5 hours. Subsequently, the first mixture is dried by evaporating water at 80° C. for 12 hours, then calcined at 500° C. for 4 hours in air atmosphere, and subsequently reduced at 500° C. for 1 hour in 10% H₂/N₂ atmosphere to obtain a dark black catalyst with ruthenium nanoparticles supported by manganese-cobalt oxide (called Ru/Mn₃Co₂Oₓ-0.2VC-H₂).

Oxidation reaction of 5-hydroxymethyl furfural is as follows: 0.0305 g of the 5-hydroxymethyl furfural, 0.0417 g of the Ru/Mn₃Co₂Oₓ-0.2VC-H₂, and 3.0576 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 31.

### Embodiments 32

A preparation method of the Mn₃Co₂Oₓ-0.2VC is the same as Embodiment 27.

A preparation of the catalyst with the noble-metal platinum nanoparticles supported by the oxide Mn₃Co₂Oₓ-0.2VC is as follows: 1.0016 g of the oxide Mn₃Co₂Oₓ-0.2VC, 0.0861 g of hexahydrate chloroplatinic acid (H₂PtCl₆·6H₂O), and 20.3453 g of deionized water are added to a beaker (100 mL) to obtain a first mixture, and the first mixture is stirred for 0.5 hours. Subsequently, the first mixture is dried by evaporating water at 80° C. for 12 hours, then calcined at 500° C. for 4 hours in air atmosphere, and subsequently reduced at 500° C. for 1 hour in 10% H₂/N₂ atmosphere to obtain a dark black catalyst with platinum nanoparticles supported by manganese-cobalt oxide (called Pt/Mn₃Co₂Oₓ-0.2VC-H₂).

Oxidation reaction of 5-hydroxymethyl furfural is as follows: 0.0314 g of the 5-hydroxymethyl furfural, 0.0414 g of Pt/Mn₃Co₂Oₓ-0.2VC-H₂, and 3.0186 g of the deionized water are added into a 25 mL autoclave, and 1 MPa air is charged into the 25 mL autoclave. Afterward, the reaction mixture is stirred at 110° C. for 2 hours. Then the 25 mL autoclave is cooled to room temperature (e.g., 20-25° C.), and the reaction solution is analyzed quantitatively. The test results are listed in Table 1 as No. 32.

### Embodiments 33

A preparation method of the Mn₁₀Co₁Oₓ-0.2VC is the same as Embodiment 7.

A preparation of the catalyst with the noble-metal platinum nanoparticles supported by the oxide Mn₁₀Co₁Oₓ-0.2VC is as follows: 1.0030 g of the oxide Mn₁₀Co₁Oₓ-0.2VC, 0.0888 g of hexahydrate chloroplatinic acid (H₂PtCl₆·6H₂O), 0.1717 g of polyvinyl pyrrolidone (PVP), and 60.0254 g of deionized water are added to a beaker (100 mL) to obtain a first mixture, and the first mixture is stirred for 0.5 hours. 0.0644 g of sodium borohydride is dissolved in 15.3153 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cobalt oxide (called Pt-PVP/Mn₁₀Co₁Oₓ-0.2VC).

Oxidation reaction of 5-hydroxymethyl furfural is as follows: 0.0304 g of the 5-hydroxymethyl furfural, 0.0407 g of the Pt-PVP/Mn₁₀Co₁Oₓ-0.2VC, and 3.0566 g of the deionized water are added into a 25 mL autoclave, and 1 MPa air is charged into the 25 mL autoclave. Afterward, the reaction mixture is stirred at 110° C. for 1 hour. Then the 25 mL autoclave is cooled to room temperature (e.g., 20-25° C.), and the reaction solution is analyzed quantitatively. The test results are listed in Table 1 as No. 33.

### Embodiment 34

A preparation of a catalyst with platinum nanoparticles supported by manganese-cerium oxide: first, 1.8940 g of manganese nitrate tetrahydrate (Mn(NO₃)₂·4H₂O), 0.3276 g of cerium nitrate hexahydrate (Ce(NO₃)₃·6H₂O), and 0.2924 g of ascorbic acid (VC) are manually ground for 5 minutes, then calcined at 200° C. for 2 hours under air atmosphere (e.g., 1 atm) to obtain an oxide Mn₁₀Ce₁Oₓ-0.2VC. Second, 1.0000 g of the Mn₁₀Ce₁Oₓ-0.2VC, 0.0889 g of hexahydrate chloroplatinic acid (H₂PtCl₆·6H₂O), and 20.1020 g of deionized water are added to a beaker (100 mL) to obtain a first mixture, and the first mixture is stirred for 0.5 hours. 0.0644 g of sodium borohydride is dissolved in 15.1459 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-cerium oxide (called Pt/Mn₁₀Ce₁Oₓ-0.2VC).

Oxidation reaction of 5-hydroxymethyl furfural is as follows: 0.0305 g of the 5-hydroxymethyl furfural, 0.0611 g of the Pt/Mn₁₀Ce₁Oₓ-0.2VC, and 3.0476 g of the deionized water are added into a 25 mL autoclave, and 1.5 MPa air is charged into the 25 mL autoclave. Afterward, the reaction mixture is stirred at 120° C. for 1.5 hours. Then the 25 mL autoclave is cooled to room temperature (e.g., 20-25° C.), and the reaction solution is analyzed quantitatively. The test results are listed in Table 1 as No. 34.

### Embodiment 35

A preparation of a catalyst with platinum nanoparticles supported by manganese-copper oxide: first, 1.8940 g of manganese nitrate tetrahydrate (Mn(NO₃)₂·4H₂O), 0.1823 g of copper nitrate trihydrate (Cu(NO₃)₂·3H₂O), and 0.2924 g of ascorbic acid (VC) are manually ground for 5 minutes, then calcined at 200° C. for 2 hours under air atmosphere (e.g., 1 atm) to obtain an oxide Mn₁₀Cu₁Oₓ-0.2VC. Second, 1.0000 g of the Mn₁₀Cu₁Oₓ-0.2VC, 0.0881 g of hexahydrate chloroplatinic acid (H₂PtCl₆·6H₂O), and 20.1020 g of deionized water are added to a beaker (100 mL) to obtain a first mixture, and the first mixture is stirred for 0.5 hours. 0.0645 g of sodium borohydride is dissolved in 15.0000 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-copper oxide (called Pt/Mn₁₀Cu₁Oₓ-0.2VC).

Oxidation reaction of 5-hydroxymethyl furfural is as follows: 0.0304 g of the 5-hydroxymethyl furfural, 0.0600 g of the Pt/Mn₁₀Cu₁Oₓ-0.2VC, and 3.0390 g of the deionized water are added into a 25 mL autoclave, and 1.5 MPa air is charged into the 25 mL autoclave. Afterward, the reaction mixture is stirred at 120° C. for 1.5 hours. Then the 25 mL autoclave is cooled to room temperature (e.g., 20-25° C.), and the reaction solution is analyzed quantitatively. The test results are listed in Table 1 as No. 35.

### Embodiment 36

A preparation of a catalyst with platinum nanoparticles supported by manganese-nickel oxide: first, 1.8940 g of manganese nitrate tetrahydrate (Mn(NO₃)₂·4H₂O), 0.2194 g of nickel nitrate hexahydrate (Ni(NO₃)₂·6H₂O), and 0.2924 g of ascorbic acid (VC) are manually ground for 5 minutes, then calcined at 200° C. for 2 hours under air atmosphere (e.g., 1 atm) to obtain an oxide Mn₁₀Ni₁Oₓ-0.2VC. Second, 1.0000 g of the Mn₁₀Ni₁Oₓ-0.2VC, 0.0885 g of hexahydrate chloroplatinic acid (H₂PtCl₆·6H₂O), and 20.1890 g of deionized water are added to a beaker (100 mL) to obtain a first mixture, and the first mixture is stirred for 0.5 hours. 0.0649 g of sodium borohydride is dissolved in 15.0000 g of the deionized water to prepare a sodium borohydride solution (a molar ratio of NaBH₄ and H₂PtCl₆·6H₂O is 10). The sodium borohydride solution is then added in drops under magnetic stirring (500 rpm) and continually stirred for 2 hours to obtain a second mixture. Finally, the second mixture is filtrated to separate the catalyst, washed with ethanol, dried under vacuum at 80° C. for 12 hours, and further ground to obtain the catalyst with platinum nanoparticles supported by manganese-nickel oxide (called Pt/Mn₁₀Ni₁Oₓ-0.2VC).

Oxidation reaction of 5-hydroxymethyl furfural is as follows: 0.0306 g of the 5-hydroxymethyl furfural, 0.0604 g of the Pt/Mn₁₀Ni₁Oₓ-0.2VC, and 3.0608 g of the deionized water are added into a 25 mL autoclave, and 1.5 MPa air is charged into the 25 mL autoclave. Afterward, the reaction mixture is stirred at 120° C. for 1.5 hours. Then the 25 mL autoclave is cooled to room temperature (e.g., 20-25° C.), and the reaction solution is analyzed quantitatively. The test results are listed in Table 1 as No. 36.

### Embodiment 37

A preparation method of the Pt/Mn₁₀Co₁Oₓ-0.2VC-LS is the same as Embodiment 14.

Oxidation reaction of 5-hydroxymethyl furfural is as follows: 0.1585 g of the 5-hydroxymethyl furfural, 0.0606 g of the Pt/Mn₁₀Co₁Oₓ-0.2VC-LS, and 2.8596 g of the deionized water are added into a 25 mL autoclave, and 0.5 MPa O₂ is charged into the 25 mL autoclave. Afterward, the reaction mixture is stirred (500 rpm) at 120° C. for 7 hours. Then the 25 mL autoclave is cooled to room temperature (e.g., 20-25° C.), and then 0.2054 g of NaHCO₃ is added into the reaction solution. Subsequently, the reaction solution is analyzed quantitatively. The test results are listed in Table 1 as No. 37.

### Embodiment 38

A preparation method of the Pt/Mn₁₀Co₁Oₓ-0.2VC-LS is the same as Embodiment 14.

Oxidation reaction of 5-hydroxymethyl furfural is as follows: 0.3031 g of the 5-hydroxymethyl furfural, 0.1215 g of the Pt/Mn₁₀Co₁Oₓ-0.2VC-LS, and 2.6992 g of the deionized water are added into a 25 mL autoclave, and 1.0 MPa O₂ is charged into the 25 mL autoclave. Afterward, the reaction mixture is stirred (500 rpm) at 120° C. for 9 hours. Then the 25 mL autoclave is cooled to room temperature (e.g., 20-25° C.), and then 0.4019 g of NaHCO₃ is added into the reaction solution. Subsequently, the reaction solution is analyzed quantitatively. The test results are listed in Table 1 as No. 38.

### Embodiment 39

A preparation method of the Pt/Mn₁₀Co₁Oₓ-0.2VC-LS is the same as Embodiment 14.

Oxidation reaction of 5-hydroxymethyl furfural is as follows: 0.4540 g of the 5-hydroxymethyl furfural, 0.1814 g of the Pt/Mn₁₀Co₁Oₓ-0.2VC-LS, and 2.5616 g of the deionized water are added into a 25 mL autoclave, and 1.5 MPa O₂ is charged into the 25 mL autoclave. Afterward, the reaction mixture is stirred (500 rpm) at 120° C. for 11 hours. Then the 25 mL autoclave is cooled to room temperature (e.g., 20-25° C.), and then 0.6017 g of NaHCO₃ is added into the reaction solution.

Subsequently, the reaction solution is analyzed quantitatively. The test results are listed in Table 1 as No. 39.

### Embodiment 40

A preparation method of the Pt/Mn₁₀Co₁Oₓ-0.2VC-LS is the same as Embodiment 14.

Oxidation reaction of 5-hydroxymethyl furfural is as follows: 0.6091 g of the 5-hydroxymethyl furfural, 0.2432 g of the Pt/Mn₁₀Co₁Oₓ-0.2VC-LS, and 2.4173 g of the deionized water are added into a 25 mL autoclave, and 2.0 MPa O₂ is charged into the 25 mL autoclave. Afterward, the reaction mixture is stirred (500 rpm) at 120° C. for 13 hours. Then the 25 mL autoclave is cooled to room temperature (e.g., 20-25° C.), and then 0.8083 g of NaHCO₃ is added into the reaction solution.

Subsequently, the reaction solution is analyzed quantitatively. The test results are listed in Table 1 as No. 40.

### Embodiment 41

A preparation method of the Pt/Mn₁₀Co₁Oₓ-0.2VC-LS is the same as Embodiment 14.

Oxidation reaction of 5-hydroxymethyl furfural is as follows: 0.7665 g of the 5-hydroxymethyl furfural, 0.3047 g of the Pt/Mn₁₀Co₁Oₓ-0.2VC-LS, and 2.2704 g of the deionized water are added into a 25 mL autoclave, and 2.5 MPa O₂ is charged into the 25 mL autoclave. Afterward, the reaction mixture is stirred (500 rpm) at 120° C. for 15 hours. Then the 25 mL autoclave is cooled to room temperature (e.g., 20-25° C.), and then 1.0024 g of NaHCO₃ is added into the reaction solution. Subsequently, the reaction solution is analyzed quantitatively. The test results are listed in Table 1 as No. 41.

### Embodiment 42

A preparation method of the Pt/Mn₁₀Co₁Oₓ-0.2VC-LS is the same as Embodiment 14.

Oxidation reaction of 5-hydroxymethyl furfural is as follows: 0.9038 g of the 5-hydroxymethyl furfural, 0.3610 g of the Pt/Mn₁₀Co₁Oₓ-0.2VC-LS, and 2.1031 g of the deionized water are added into a 25 mL autoclave, and 3.0 MPa O₂ is charged into the 25 mL autoclave. Afterward, the reaction mixture is stirred (500 rpm) at 120° C. for 17 hours. Then the 25 mL autoclave is cooled to room temperature (e.g., 20-25° C.), and then 1.2005 g of NaHCO₃ is added into the reaction solution. Subsequently, the reaction solution is analyzed quantitatively. The test results are listed in Table 1 as No. 42.

### Embodiment 43

A preparation method of the Pt/Mn₁₀Co₁Oₓ-0.2VC-LS is the same as Embodiment 14.

Oxidation reaction of 5-hydroxymethyl furfural is as follows: 1.0556 g of the 5-hydroxymethyl furfural, 0.4211 g of the Pt/Mn₁₀Co₁Oₓ-0.2VC-LS, and 1.9623 g of the deionized water are added into a 25 mL autoclave, and 3.5 MPa 02 is charged into the 25 mL autoclave. Afterward, the reaction mixture is stirred (500 rpm) at 120° C. for 19 hours. Then the 25 mL autoclave is cooled to room temperature (e.g., 20-25° C.), and then 1.4100 g of NaHCO₃ is added into the reaction solution. Subsequently, the reaction solution is analyzed quantitatively. The test results are listed in Table 1 as No. 43.

### Embodiment 44

A preparation method of the Pt/Mn₁₀Co₁Oₓ-0.2VC-LS is the same as Embodiment 14.

Oxidation reaction of 5-hydroxymethyl furfural is as follows: 1.2073 g of the 5-hydroxymethyl furfural, 0.4802 g of the Pt/Mn₁₀Co₁Oₓ-0.2VC-LS, and 1.8112 g of the deionized water are added into a 25 mL autoclave, and 4.0 MPa 02 is charged into the 25 mL autoclave. Afterward, the reaction mixture is stirred (500 rpm) at 120° C. for 21 hours. Then the 25 mL autoclave is cooled to room temperature (e.g., 20-25° C.), and then 1.6000 g of NaHCO₃ is added into the reaction solution. Subsequently, the reaction solution is analyzed quantitatively. The test results are listed in Table 1 as No. 44.

### Embodiment 45

A preparation method of the Pt/Mn₁₀Co₁Oₓ-0.2VC-LS is the same as Embodiment 14.

Oxidation reaction of 5-hydroxymethyl furfural is as follows: 0.0492 g of the crude 5-hydroxymethyl furfural purchased from Zhongke Guosheng Technology Co., Ltd. (61 wt %, Hangzhou, China), 0.0253 g of the Pt/Mn₁₀Co₁Oₓ-0.2VC-LS, and 3.0210 g of the deionized water are added into a 25 mL autoclave, and 0.5 MPa 02 is charged into the 25 mL autoclave. Afterward, the reaction mixture is stirred (500 rpm) at 120° C. for 6 hours. Then the 25 mL autoclave is cooled to room temperature (e.g., 20-25° C.), and then 0.0401 g of NaHCO₃ is added into the reaction solution.

Subsequently, the reaction solution is analyzed quantitatively. The test results are listed in Table 1 as No. 45.

### Embodiment 46

A preparation method of the Pt/Mn₁₀Co₁Oₓ-0.2VC-LS is the same as Embodiment 14.

Oxidation reaction of 5-hydroxymethyl furfural is as follows: 0.2501 g of the crude 5-hydroxymethyl furfural purchased from Zhongke Guosheng Technology Co., Ltd. (61 wt %, Hangzhou, China), 0.1266 g of the Pt/Mn₁₀Co₁Oₓ-0.2VC-LS, and 2.7831 g of the deionized water are added into a 25 mL autoclave, and 1.0 MPa O₂ is charged into the 25 mL autoclave. Afterward, the reaction mixture is stirred (500 rpm) at 120° C. for 8 hours. Then the 25 mL autoclave is cooled to room temperature (e.g., 20-25° C.), and then 0.2001 g of NaHCO₃ is added into the reaction solution. Subsequently, the reaction solution is analyzed quantitatively. The test results are listed in Table 1 as No. 46.

### Embodiment 47

A preparation method of the Pt/Mn₁₀Co₁Oₓ-0.2VC-LS is the same as Embodiment 14.

Oxidation reaction of 5-hydroxymethyl furfural is as follows: 0.4968 g of the crude 5-hydroxymethyl furfural purchased from Zhongke Guosheng Technology Co., Ltd. (61 wt %, Hangzhou, China), 0.2405 g of the Pt/Mn₁₀Co₁Oₓ-0.2VC-LS, and 2.5475 g of the deionized water are added into a 25 mL autoclave, and 1.5 MPa O₂ is charged into the 25 mL autoclave. Afterward, the reaction mixture is stirred (500 rpm) at 120° C. for 10 hours. Then the 25 mL autoclave is cooled to room temperature (e.g., 20-25° C.), and then 0.4100 g of NaHCO₃ is added into the reaction solution. Subsequently, the reaction solution is analyzed quantitatively. The test results are listed in Table 1 as No. 47.

### Embodiment 48

A preparation method of the Pt/Mn₁₀Co₁Oₓ-0.2VC-LS is the same as Embodiment 14.

Oxidation reaction of 5-hydroxymethyl furfural is as follows: 0.7377 g of the crude 5-hydroxymethyl furfural purchased from Zhongke Guosheng Technology Co., Ltd. (61 wt %, Hangzhou, China), 0.3601 g of the Pt/Mn₁₀Co₁Oₓ-0.2VC-LS, and 2.2885 g of the deionized water are added into a 25 mL autoclave, and 2.0 MPa O₂ is charged into the 25 mL autoclave. Afterward, the reaction mixture is stirred (500 rpm) at 120° C. for 13 hours. Then the 25 mL autoclave is cooled to room temperature (e.g., 20-25° C.), and then 0.6094 g of NaHCO₃ is added into the reaction solution. Subsequently, the reaction solution is analyzed quantitatively. The test results are listed in Table 1 as No. 48.

TABLE 1
Test Results

| | | HMF | | | |
|---|---|---|---|---|---|
| | | conversion | Yield (%) | | |
| No. | Catalyst | rate (%) | DFF | FFCA | FDCA |
| 1 | Pt/MnOₓ | 94.9 | 13.5 | 58.1 | 14.6 |
| 2 | Pt/CoOₓ | 100 | 0.0 | 37.0 | 41.7 |
| 3 | Pt/MnOₓ-0.2VC | 100 | 0.0 | 14.1 | 64.5 |
| 4 | Pt/CoOₓ-0.2VC | 100 | 0.8 | 46.8 | 32.8 |
| 5 | Pt/Mn₁₄Co₁Oₓ-0.2VC | 99.2 | 0.8 | 26.4 | 67.7 |
| 6 | Pt/Mn₁₂Co₁Oₓ-0.2VC | 99.4 | 0.6 | 22.2 | 71.3 |
| 7 | Pt/Mn₁₀Co₁Oₓ-0.2VC | 100 | 0.0 | 11.9 | 83.1 |
| 8 | Pt/Mn₈Co₁Oₓ--0.2VC | 99.2 | 0.6 | 23.2 | 67.5 |
| 9 | Pt/Mn₆Co₁Oₓ--0.2VC | 99.3 | 0.9 | 24.3 | 64.1 |
| 10 | Pt/Mn₄Co₁Oₓ--0.2VC | 99.3 | 0.5 | 26.4 | 62.5 |
| 11 | Pt/Mn₃Co₁Oₓ--0.2VC | 99.3 | 0.9 | 33.6 | 55.1 |
| 12 | Pt/Mn₁₀Co₁Oₓ-0.2VC | 100 | 0.0 | 2.5 | 95.3 |
| 13 | Pt/Mn₁₀Co₁Oₓ-0.2VC | 100 | 0.0 | 3.3 | 95.8 |
| 14 | Pt/Mn₁₀Co₁Oₓ-0.2VC-LS | 100 | 0.0 | 12.1 | 84.9 |
| 15 | Pt/Mn₁₀Co₁Oₓ-0.2VC | 98.9 | 1.0 | 28.7 | 67.3 |
| 16 | Pt/Mn₁₀Co₁Oₓ | 91.1 | 10.0 | 50.0 | 27.9 |
| 17 | Pt/Mn₁₀Co₁Oₓ-0.1VC | 99.3 | 0.6 | 27.1 | 69.4 |
| 18 | Pt/Mn₁₀Co₁Oₓ-0.3VC | 100 | 0.0 | 6.9 | 82.6 |
| 19 | Pt/Mn₁₀Co₁Oₓ-0.4VC | 100 | 0.0 | 9.9 | 78.8 |
| 20 | Pt/Mn₁₀Co₁Oₓ-0.2VC- | 100 | 0.0 | 15.1 | 77.1 |
| | 300° C. | | | | |
| 21 | Pt/Mn₁₀Co₁Oₓ-0.2VC- | 100 | 0.0 | 17.9 | 71.3 |
| | 400° C. | | | | |
| 22 | Pt/Mn₁₀Co₁Oₓ-0.2VC- | 100 | 0.0 | 14.9 | 71.2 |
| | 500° C. | | | | |
| 23 | Pt(1.1 wt %)/Mn₁₀Co₁Oₓ- | 99.5 | 0.0 | 20.1 | 71.8 |
| | 0.2VC-200° C. | | | | |
| 24 | Pt(1.8 wt %)/Mn₁₀Co₁Oₓ- | 100 | 0.0 | 10.6 | 76.5 |
| | 0.2VC-200° C. | | | | |
| 25 | Pt(3.3 wt %)/Mn₁₀Co₁Oₓ- | 89.1 | 8.1 | 47.4 | 30.0 |
| | 0.2VC-200° C. | | | | |
| 26 | Pt(4.4 wt %)/Mn₁₀Co₁Oₓ- | 98.1 | 2.2 | 41.9 | 52.9 |
| | 0.2VC-200° C. | | | | |
| 27 | Ru/Mn₃Co₂Oₓ-0.2VC | 100 | 6.3 | 14.6 | 53.7 |
| 28 | Pt/Mn₃Co₂Oₓ-0.2VC | 100 | 7.2 | 16.9 | 72.3 |
| 29 | Au/Mn₃Co₂Oₓ-0.2VC | 48.4 | 9.4 | 20.1 | 8.7 |
| 30 | Pd/Mn₃Co₂Oₓ-0.2VC | 80.0 | 2.9 | 31.6 | 24.5 |
| 31 | Ru/Mn₃Co₂Oₓ-0.2VC-H₂ | 100 | 21.4 | 43.3 | 12.4 |
| 32 | Pt/Mn₃Co₂Oₓ-0.2VC-H₂ | 100 | 10.9 | 47.5 | 17.7 |
| 33 | Pt-PVP/Mn₁₀Co₁Oₓ-0.2VC | 100 | 0.0 | 12.3 | 81.1 |
| 34 | Pt/Mn₁₀Ce₁Oₓ-0.2VC | 100 | 0.0 | 6.3 | 87.7 |
| 35 | Pt/Mn₁₀Cu₁Oₓ-0.2VC | 76.6 | 5.1 | 23.2 | 24.2 |
| 36 | Pt/Mn₁₀Ni₁Oₓ-0.2VC | 100 | 0.0 | 7.2 | 87.1 |
| 37 | Pt/Mn₁₀Co₁Oₓ-0.2VC-LS | 100 | 0.0 | 0.0 | 93.5 |
| 38 | Pt/Mn₁₀Co₁Oₓ-0.2VC-LS | 100 | 0.0 | 0.0 | 92.9 |
| 39 | Pt/Mn₁₀Co₁Oₓ-0.2VC-LS | 100 | 0.0 | 0.0 | 93.6 |
| 40 | Pt/Mn₁₀Co₁Oₓ-0.2VC-LS | 100 | 0.0 | 0.0 | 95.2 |
| 41 | Pt/Mn₁₀Co₁Oₓ-0.2VC-LS | 100 | 0.0 | 0.0 | 94.5 |
| 42 | Pt/Mn₁₀Co₁Oₓ-0.2VC-LS | 100 | 0.0 | 0.0 | 94.9 |
| 43 | Pt/Mn₁₀Co₁Oₓ-0.2VC-LS | 100 | 0.0 | 0.0 | 91.7 |
| 44 | Pt/Mn₁₀Co₁Oₓ-0.2VC-LS | 100 | 0.0 | 0.0 | 83.2 |
| 45 | Pt/Mn₁₀Co₁Oₓ-0.2VC-LS | 100 | 0.0 | 0.0 | 93.7 |
| 46 | Pt/Mn₁₀Co₁Oₓ-0.2VC-LS | 100 | 0.0 | 0.0 | 92.6 |
| 47 | Pt/Mn₁₀Co₁Oₓ-0.2VC-LS | 100 | 0.0 | 0.0 | 91.8 |
| 48 | Pt/Mn₁₀Co₁Oₓ-0.2VC-LS | 100 | 0.0 | 21.9 | 73.0 |

Notes:
2,5-Diformylfuran (DFF); 5-formyl-2-furancarboxylic acid (FFCA)

The catalytic system is compared with the existing catalytic systems, results are shown in Table 2:

TABLE 2
Comparison between the catalytic system and other catalytic systems

| | | | | | | Generation |
|---|---|---|---|---|---|---|
| | | | | HMF | | rate |
| | | | | Conversion | FDCA | [mol_{FDCA} |
| | Catalyst and | Solvent, | reaction | rate | Yield | molₘₑₜₐₗ_{^{−1}} |
| NO. | amount ^{a} | alkali | conditions | (%) | (%) | h⁻¹] ^{c} |
| Embodiment | Pt/Mn₁₀Co₁Oₓ—0.2VC, | H₂O, base- | 1.5 hours, | 100 | 95.3 | 24.8 |
| 12 | 39 | free | 120° C., 15 | | | |
| | | | bar Air | | | |
| Embodiment | Pt/Mn₁₀Co₁Oₓ—0.2VC, | H₂O, base- | 1 hour, | 100 | 95.8 | 37.4 |
| 13 | 39 | free | 120° C., 2 | | | |
| | | | bar O₂ | | | |
| [1] | Ru(4%)/MnCo₂O₄, | H₂O, base- | 10 hours, | 100 | 99 | 3.4 |
| | 34 | free | 120° C., 24 | | | |
| | | | bar Air | | | |
| [2] | Ru/CTF, 40 | H₂O, base- | 3 hours, | 100 | 78 | 10.4 |
| | | free | 140° C., 20 | | | |
| | | | bar Air | | | |
| [3] | Au/0.4MgF₂—0.6MgO, | H₂O, base- | 2 hours, | 98 | 63 | 15.8 |
| | 50 | free | 110° C., 26 | | | |
| | | | bar Air | | | |
| [4] | Ni_{0.5}Pd_{0.5}/Mg(OH)₂, | H₂O, base- | 10 hours, | 100 | 98 | 3.9 |
| | 40 | free | 100° C., 1 | | | |
| | | | bar Air | | | |
| [5] | Ru/Mn₆Ce₁O_{Y}, 80 | H₂O, base- | 15 hours, | 100 | 100 | 5.3 |
| | | free | 150° C., 10 | | | |
| | | | bar O₂ | | | |
| [6] | Au₁Pd₁/pBNC-30% | H₂O, base- | 48 hours, | 99.8 | 93.9 | 1.1 |
| | HNO₃, 55 | free | 100° C., 2.0 | | | |
| | | | MPa O₂ | | | |
| [7] | Pt/HT, 100 | H₂O, base- | 14 hours, | 100 | 97 | 6.9 |
| | | free | 95° C., 5 | | | |
| | | | bar O₂ | | | |
| [8] | Pt/C—O—Mg, 50 | H₂O, base- | 12 hours, | 100 | 97 | 4.1 |
| | | free | 110° C., 10 | | | |
| | | | bar O₂ | | | |
| [9] | Pt/ZrO₂-ALD, 73 | H₂O, base- | 12 hours, | 100 | 97 | 5.9 |
| | | free | 100° C., 4 | | | |
| | | | bar O₂ | | | |
| [10] | Pt/C-EDA-4.1, 50 | H₂O, base- | 12 hours, | >99 | 96 | 4.0 |
| | | free | 110° C., 10 | | | |
| | | | bar O₂ | | | |

Note:
\textcircled{1} ^{a} the amount of the catalyst, refers to a molar ratio of the noble metal and HMF.
^{b} refers to an HMF concentration.
^{c} generation rate, refers to a molar amount of a target product obtained per mole amount of the noble metal per hour.
\textcircled{2} The aforementioned other catalytic systems are disclosed in the following references:
[1] Green Chem., 2017, 19, 1619-1623.
[2] ChemSusChem, 2015, 8, 3832-3838.
[3] ACS Sustainable Chem. Eng., 2018, 6, 16332-16340.
[4] Inorg. Chem. Front., 2017, 4, 871-880.
[5] Journal of Catalysis., 368, (2018) 53-68.
[6] ChemSusChem, 2022, e202201041.
[7] ChemCatChem, 7 (2015) 2853-2863.
[8] Green Chem., 18 (2016) 1597-1604.
[9] Appl. Catal., A 555 (2018) 98-107.
[10] Appl. Catal., A 526 (2016) 1-8.

Referring to the Table 2, it can be seen that other reported Pt-based catalysts usually require a reaction time of 9-14 hours to achieve similar catalytic effects under the base-free condition. The catalyst of the present disclosure is currently the most active Pt-based catalyst under the base-free condition.

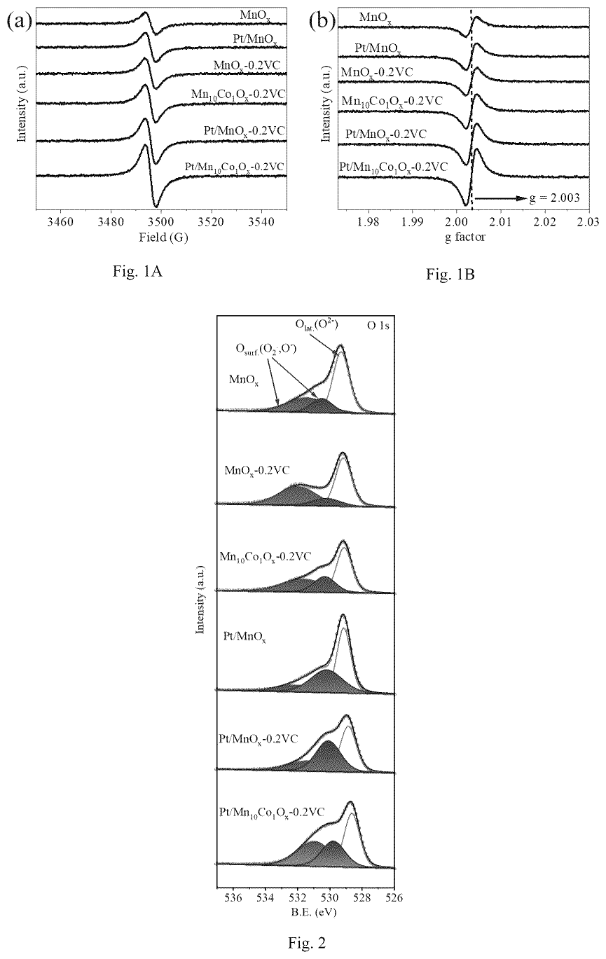

TABLE 3
A relative content of oxygen vacancies on a surface of the
catalysts respectively obtained from EPR electron
paramagnetic resonance spectrum^{b} in FIGS. 1A and 1B
and high-resolution XPS spectrum^{a} in FIG. 2

| | | Relative content | Specific content of |
|---|---|---|---|
| | | of oxygen | oxygen vacancies |
| No. | Catalysts | vacancies (%)^{a} | ((spins · g⁻¹)^{b} |
| 1 | MnOx | 46 | 1.567 * 10¹⁶ |
| 2 | Pt/MnOₓ | 51 | 2.098 * 10¹⁶ |
| 3 | MnOₓ-0.2VC | 54 | 2.140 * 10¹⁶ |
| 4 | Mn₁₀Co₁Oₓ-0.2VC | 56 | 2.739 * 10¹⁶ |
| 5 | Pt/MnOₓ-0.2VC | 61 | 3.288 * 10¹⁶ |
| 6 | Pt/Mn₁₀Co₁Oₓ-0.2VC | 64 | 4.476 * 10¹⁶ |

Referring to Table 3, it can be seen that an addition of vitamin C additives can significantly increase the content of the oxygen vacancies on the surface of Mn-based oxide carriers.

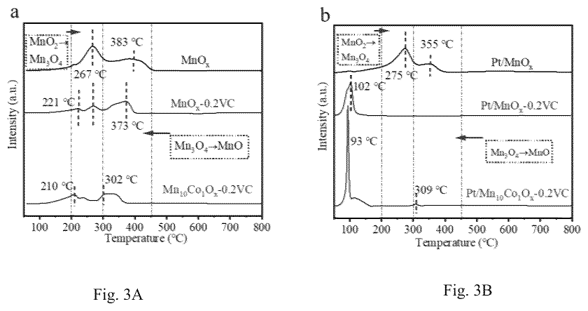

An H₂-TPR spectrum in FIG. 3A indicates that an introduction of the vitamin C additives enables the Mn-based oxide carriers to be easily reduced, that is, lattice oxygen is more likely lost, and an activity of the lattice oxygen becomes higher. In addition, the H₂-TPR spectrum in FIG. 3B indicates that a lower reduction peak appears after metal platinum is loaded on the Mn-based oxide carrier, indicating a formation of highly active lattice oxygen sites on surfaces of the Pt/Mn₁₀Co₁Oₓ-0.2VC and the Pt/MnOₓ-0.2VC.

The aforementioned embodiments are merely used to illustrate the technical solution of the present disclosure instead of a limitation of the technical solution of the present disclosure. Although the present disclosure has been described in detail in combination with the aforementioned embodiments, it should be understood for person of skill in the art, the technical solution described in the aforementioned embodiments can still be modified, or some or all of the technical features can still be equivalently replaced. These modifications or replacements are made without resulting in a substance of the corresponding technical solution departing from the scope of the various embodiments of the present disclosure.

What is claimed is:

1. A method for preparing 2,5-furanedicarboxylic acid by catalyzing 5-hydroxymethylfurfural in a base-free condition, comprising:
synthesizing the 2,5-furanedicarboxylic acid by catalyzing the 5-hydroxymethylfurfural using a catalyst in the base-free condition with a solvent of water using air or oxygen as an oxygen source, and a reaction time is 0.5-21 hours; and
the catalyst is a metal catalyst supported by an Mn-based bimetallic oxide enriched with oxygen vacancies, a formula of the catalyst is A/MnₐB_{b}Oₓ-yVC, wherein:
A is at least one of Pt, Ru, Pd, or Au,
B is at least one of Co, Ce, Cu, or Ni,
a mole ratio of a and b is 1.5-14, and
y=0.0-0.4.

2. The method according to claim 1, wherein:
a reaction pressure is 0.2-4.0 MPa,
a temperature of a reaction autoclave for the method is 80-130° C., and
the reaction time is 1-21 hours.

3. The method according to claim 1, wherein preparing the catalyst comprises the following steps:
1) mixing and grinding a precursor manganese nitrate, metal nitrate, and ascorbic acid, then calcining at 200-500° C. for 2 hours to obtain a carrier with the Mn-based bimetallic oxide enriched with oxygen vacancies, wherein the metal nitrate is at least one of cobalt nitrate, cerium nitrate, copper nitrate, or nickel nitrate, a molar ratio of the precursor manganese nitrate and the metal nitrate is 1.5-14:1, and a molar ratio of the ascorbic acid and a sum of the precursor manganese nitrate and the metal nitrate is 0-0.4:1; and
2) adding at least one of hexahydrate chloroplatinic acid, trihydrate ruthenium chloride, palladium chloride, trihydrate chloroauric acid and the carrier with the Mn-based bimetallic oxide enriched with oxygen vacancies to deionized water, stirring to be dispersed to even, and reducing to obtain the catalyst.

4. The method according to claim 3, wherein:
in the step 2), the reducing to obtain the catalyst comprises adding a sodium borohydride solution, continually stirring for 2 hours, then filtering, and drying the catalyst.

5. The method according to claim 3, wherein:
the reducing to obtain the catalyst in the step 2) comprises:
drying by evaporating water,
then calcinating at 500° C. for 4 hours, and
then reducing at 500° C. in a hydrogen atmosphere for 1 hour.

6. A metal catalyst supported by an Mn-based bimetallic oxide enriched with oxygen vacancies, wherein:
a metal of the metal catalyst is A, and
a formula of the metal catalyst is A/MnₐB_{b}Oₓ-yVC, wherein:
A is at least one of Pt, Ru, Pd, or Au,
B is at least one of Co, Ce, Cu, or Ni,
a mole ratio of a and b is 1.5-14, and
y=0.0-0.4.

7. An application of the metal catalyst supported by the Mn-based bimetallic oxide enriched with the oxygen vacancies according to claim 6, comprising:
preparing 2,5-furanedicarboxylic acid by catalyzing 5-hydroxymethylfurfural using the metal catalyst supported by the Mn-based bimetallic oxide enriched with the oxygen vacancies.

8. The application according to claim 7, wherein:
the preparing 2,5-furanedicarboxylic acid by catalyzing 5-hydroxymethylfurfural using the metal catalyst supported by the Mn-based bimetallic oxide enriched with the oxygen vacancies comprises:
mixing the 5-hydroxymethylfurfural and water solvent and placing in a reaction kettle;
adding the metal catalyst under a base-free condition; and
sealing the reaction kettle, and filling with air or oxygen, wherein a pressure is 0.2-4.0 MPa, a temperature of the reaction kettle is 80-130° C., and a reaction time is 0.5-21 hours.

9. The application according to claim 8, wherein the pressure is 0.5-2.5 MPa, and the reaction time 0.5-2 hours.

10. The method according to claim 1, wherein the reaction time is 0.5-2 hours.

11. The method according to claim 1, wherein y is 0.1-0.4.

12. The method according to claim 1, wherein a loading amount of A is 1-5 wt %.

13. The metal catalyst supported by the Mn-based bimetallic oxide enriched with the oxygen vacancies according to claim 6, wherein a loading amount of A is 1-5 wt %.

14. The metal catalyst supported by the Mn-based bimetallic oxide enriched with the oxygen vacancies according to claim 6, wherein y is 0.1-0.4.
